(19) United States
(12) Patent Application Publication
(10) Pub. No.: US 2011/0160431 A1
(43) Pub. Date: Jun. 30, 2011

(54) PRODUCTION OF PEPTIDES CONTAINING POLY-GLY SEQUENCES USING FMOC CHEMISTRY

(75) Inventors: Avi Tovi, Zurit (IL); Chaim Eidelman, Oshrat (IL); Shimon Shushan, Karmiel (IL); Hagi Alon, Kibbutz Yehiam (IL); Alexander Ivchenko, Keryat Ata (IL); Leah Bar-0z, Kiryat Bialik (IL)

(73) Assignee: NOVETIDE, LTD., Haifa Bay (IL)

(21) Appl. No.: 12/996,770

(22) PCT Filed: Mar. 29, 2010
(86) PCT No.: PCT/US10/29054

§ 371 Date: Jan. 6, 2011

Related U.S. Application Data
(60) Provisional application No. 61/166,937, filed on Apr. 6, 2009.

(51) Int. Cl. C07K 7/08; C07K 1/06

(52) U.S. Cl. 530/326; 530/334

(57) ABSTRACT

A peptide containing a poly-Gly sequence, such as bivalirudin, can be prepared in a purified form in which low amounts of GIy deletion or GIy addition byproducts are present. A protected poly-Gly-containing peptide is attached to a resin using Fmoc-Gly-GIy-OH units for assembly of the poly-Gly segment. The protected peptide is then cleaved from the resin with an acidic composition to produce an unprotected or semi-protected crude peptide, which can then be isolated from acidic composition.

### CROSS-REFERENCE TO RELATED APPLICATIONS

This application claims the benefit of U.S. Provisional Patent Application Ser. No. 61/166,937, filed Apr. 6, 2009, which is incorporated herein by reference.

### FIELD OF THE INVENTION

The present invention encompasses a new synthetic approach for assembly of poly-Gly sequences in peptide synthesis using Fmoc chemistry. Some embodiments are directed to the production of highly pure Bivalirudin.

### BACKGROUND OF THE INVENTION

Peptide synthesis may be carried out either by solid-phase synthesis (SPPS) or by solution-phase synthesis and generally proceeds from the C-terminus to N-terminus.

The synthesis may be performed according to two main routes: a) a sequential route comprising a stepwise addition of a given amino acid to a growing peptide, or b) a fragment condensation route comprising combining several short fragments to form a desired peptide sequence.

Solution phase synthesis is usually based on fragment condensation; solid phase synthesis is usually based on sequential addition of α-amino and side-chain protected amino acid residues to an insoluble polymeric support, the resin. The acid-labile Boc group or the basic-labile Fmoc-group are commonly used for the N-α-protection. Following removal of this protecting group, the successive protected amino acid is attached to the growing peptide-resin using either a coupling reagent or pre-activated protected amino acid derivative. Removal of the Boc protecting group can be performed by treatment with trifluoroacetic acid (TFA) and removal of the Fmoc protecting group can be performed by treatment with piperidine.

The desired peptide is finally obtained attached to the resin, via a linker, through its C-terminus. It may be cleaved to yield a peptide acid or amide, depending on the linking agent used. This final cleavage of the peptidyl resin and side-chain deprotection requires a strong acid, such as hydrogen fluoride (HF) or TFMSA in the case of Boc chemistry, and TFA in Fmoc chemistry.

The development of Fmoc chemistry arose from the concern that repetitive TFA acidolysis in Boc-group deprotection would lead to alteration of sensitive peptide bonds as well as acid catalyzed side reactions. In Fmoc synthesis, the growing peptide is subjected to mild base treatment for protecting groups cleavage and TFA is required only for the final cleavage of the peptidyl resin. In the Boc synthesis the cleavage of the resin and side-chain deprotection requires the use of dangerous HF and therefore unique equipment.

Each peptide is characterized by its own specific sequence of amino acids. Some peptides contain short repeats of the same amino acid, such as the sequence of Atrial natriuretic factor containing Gly-Gly fragment (H-Ser-Leu-Arg-Arg-Ser-Ser-Cys-Phe-Gly-Gly-Arg-Met-Asp-Arg-Ile-Gly-Ala-Gln-Ser-Gly-Leu-Gly-Cys-Asn-Ser-Phe-Arg-Tyr-OH), C-peptide containing Gly-Gly-Gly sequence (H-Arg-Arg-Glu-Ala-Glu-Asp-Leu-Gln-Val-Gly-Gln-Val-Glu-Leu-Gly-Gly-Gly-Pro-Gly-Ala-Gly-Ser-Leu-Gln-Pro-Leu-Ala-Leu-Glu-Gly-Ser-Leu-Gln-Lys-Arg-OH), Dynorphin B containing Gly-Gly sequence (H-Tyr-Gly-Gly-Phe-Leu-Arg-Arg-Gln-Phe-Lys-Val-Val-Thr-OH), Endorphin alfa containing Gly-Gly sequence (H-Tyr-Gly-Gly-Phe-Met-Thr-Ser-Glu-Lys-Ser-Gln-Thr-Pro-Leu-Val-Thr-OH), Diapep277 containing Gly-Gly-Gly sequence (H-Val-Leu-Gly-Gly-Gly-Val-Ala-Leu-Leu-Arg-Val-Ile-Pro-Ala-Leu-Asp-Ser-Leu-Thr-Pro-Ala-Asn-Glu-Asp-OH), Terlipressin containing Gly-Gly-Gly sequence (H-Gly-Gly-Gly-Cys-Tyr-Phe-Gln-Asn-Cys-Pro-Lys-Gly-NH₂), Exenatide containing Gly-Gly sequence (H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂), and Bivalirudin containing four Gly in the molecule. Bivalirudin (Hirulog-8) is a 20 amino acid peptide with the following primary structure: H-D-Phe-Pro-Arg-Pro-Gly-Gly-Gly-Gly-Asn-Gly-Asp-Phe-Glu-Glu-Ile-Pro-Glu-Glu-Tyr-Leu-OH.

Bivalirudin is a specific and reversible direct thrombin inhibitor, indicated as an anticoagulant in patients with unstable angina undergoing percutaneous transluminal coronary angioplasty (PTCA).

Preparation of Bivalirudin has been Described Using a Sequential Approach on a solid phase applying Boc-protected amino acids (WO 91/02750). The synthesis was performed using a Boc-L-Leucine-O-divinylbenzene resin. Additional t-Boc-amino acids used included Boc-O-2,6-dichlorobenzyl tyrosine, Boc-L-glutamic acid (7-benzyl ester), Boc-L-proline, Boc-L-isoleucine, Boc-L-phenylalanine, Boc-L-aspartic acid (B-benzyl ester), Boc-glycine, Boc-L-asparagine, Boc-L-phenylalanine, and Boc-L-arginine. In order to achieve higher yields in synthesis, the (Gly)4 linker segment was attached in two cycles of manual addition of Boc-glycylglycine. After completion of the synthesis, the peptide was fully deprotected and uncoupled from the divinylbenzene resin by treatment with anhydrous HF: p-cresol: ethylmethyl sulfate (10:1:1, v/v/v).

As discussed above, due to the properties of the Gly amino acid, its introduction during the peptide assembly does not require special concern. The same is generally true in the synthesis of multiple-Gly sequences, for example, in the synthesis of the four-Gly sequence during the Bivalirudin synthesis using Boc chemistry. This synthesis may be carried out either sequentially (Okayama et al.; 1996, Chem. Pharm. Bull. 44:1344-1350 and Steinmetzer at al.; 1999, Eur. J. Biochem. 265:595-605) or in two cycles of introducing a Boc-Gly-Gly-OH unit (WO 91/02750).

Introduction of a Boc-Gly-Gly-OH unit could also be found in other examples such as preparation of N-di- and tripeptidyl derivatives of antibiotics (Topliss, John G.; Afonso, Adriano, U.S. Pat. No. 4,169,141) or preparation of triglycyl-lysine vasopressin (Abraham, Nedumparambil A.; Immer, Hans U.; Sestanj, Kazimir; U.S. Pat. No. 4,093,610).

An example in which Fmoc-Gly-Gly-OH was used may be found in the preparation of spacers or linkages between various organic compounds such as Mass Tags (US application 179,060) and conjugation of selenols (Ide, Nathan D.; Galonic, Danica P.; van der Donk, Wilfred A.; Gin, David Y.; Synlett (2005), (13), 2011-2014), or in peptide synthesis (Scott, William L.; Alsina, Jordi; Kennedy, Joseph H.; O'Donnell, Martin J.; Organic Letters (2004), 6(10), 1629-1632).

In a peptide sequence each amino acid contains at least one amino group (N-terminus) and at least one carboxy group (C-terminus). The main difference between various amino acids is due to their side chain groups that actually define their chemical and physical characteristics. The simplest amino acid is Gly as it contains no side chain on its backbone. It also contains no chiral atom and therefore its introduction into the peptide sequence is free from racemization.

Gly may be introduced sequentially by regular coupling methods using Boc-Gly, Fmoc-Gly or other N-protected Gly derivatives. The same approach may also be generally carried out for the addition of several Gly units sequence, as described in sequential synthesis of Bivalirudin.

It is known that during the synthesis of peptides using the sequential route, by-products may be obtained due to the addition or deletion of one amino acid. The addition of an amino acid may occur if the N-α-protection is absent, therefore exposing the growing peptide to the addition of two amino acids instead of one. A deletion of amino acid may occur due to an incomplete reaction wherein the required amino acid is not properly added to a specific growing peptide.

This formation of by-products having an addition or deletion of one amino acid is even more significant when synthesizing a peptide having short repeats of the same amino acid. It may be readily understood that in a case where short repeats of the same amino acid are present in the peptide, the amount of a certain impurity, either addition or deletion, will be higher. For example, if a certain peptide contains a sequence of 4 proline amino acids then the deletion of each of the amino acids will lead to the same by-product, a peptide containing 3 proline amino acids instead of four. The same goes for an addition byproduct containing 5 proline amino acids. Therefore, although the occurrence of each addition/deletion will not change, the by-products which will be formed will accumulate to a higher amount.

These byproducts are usually easily detected and may be isolated from the peptide due to their different weight and properties. However, this is not the case for poly-Gly containing peptides. Since glycine is the simplest amino acid, it is difficult to distinguish between the peptide and the byproducts containing an addition of Glycine ([+Gly]) or deletion of Glycine ([des-Gly]) making it hard to identify and isolate the impurities from the peptide.

Previously described routes for the synthesis of Bivalirudin have failed to identify methods for the preparation of a highly pure product having a low amount of by-products containing an addition of Glycine ([+Gly]-Bivalirudin) or deletion of Glycine ([des-Gly]-Bivalirudin). Purity of the active compound is an extremely important parameter, especially for the products used as APIs (active pharmaceutical ingredients). Various grades of purity of the same product are possible at the end of the production process. In general, the purity of the product depends on the chemistry and various process-related parameters of the production process to provide a high quality product. In the case of peptide products the situation is even more complicated as peptides are complex and sensitive molecules. They are produced by multi-step processes applying an extensive variety of starting materials and are potentially contaminated due to the many possible side reactions, which are part of peptide chemistry.

The literature in the art lacks discussion of specific problems or restrictions regarding the assembly of multiple-Gly sequences. Moreover, it seems that in cases where such sequences were synthesized using Boc-Gly-Gly-OH or Fmoc-Gly-Gly-OH units, it was only for the purpose of achieving better yield or because it was an easier way to insert the specific sequence. None of these examples report the formation of specific impurities or any problem in the purification of the final product.

To make this point even more clear it is interesting to examine the example of synthesis of Heterotrimeric collagen peptides containing multiple Gly-Pro-Hyp fragments and also one Gly-Gly segment (Ottl, Johannes; Musiol, Hans Jurgen; Moroder, Luis; Journal of Peptide Science (1999), 5(2), 103-110). The crude products resulting from the stepwise elongation procedure and from the use of Fmoc-Gly-Pro-Hyp(tBu)-OH clearly revealed large amounts of impurities as a result of incomplete couplings of amino acids as well as diketopiperazine formation due to cleavage of Gly-Pro units from the growing peptide chain. It is reported that by using the Fmoc-Pro-Hyp-Gly-OH synthon, the quality of the crude products was significantly improved. There is no description, however, of any synthetic problem arising from the use of Gly-Gly synthesis in this sequence and thus it could be concluded that the use of Fmoc-Gly-Gly-OH was only to shorten the reaction cycle and not for any other reason, such as prevention of formation of any specific impurity.

The preparation of Bivalirudin using Fmoc chemistry by sequential method on CTC resin was recently reported (WO 2006/045503 A1). In this publication there is a remark that “instead of coupling both side chain and N-α protected amino acids, N-α-alkyl protected dipeptide modules may be used for coupling during linear synthesis; such dipeptides have secondary structure disrupting effect, easing yield and purity of the synthesis. For example, Fmoc-Gly-(N-Hmb)Gly-OH and Fmoc-Gly-(N-Dmb)Gly-OH are commercially available from EMD Bioscences (Novabiochem). It is to be understood that such N-alkyl groups are not considered protecting groups in the sense of the present invention, hence their use or presence is optional and not excluded by the structure of formula I”. The ease in purity which is mentioned relates to reduction of impurities of folded peptides, aggregates or peptides which formed hydrogen bonds with adjacent peptides.

As could be understood from the publication above, the sequence of Gly-Gly-Gly-Gly in Bivalirudin could be obtained using Fmoc-Gly-(N-Hmb)Gly-OH or Fmoc-Gly-(N-Dmb)Gly-OH units instead of Fmoc-Gly-OH. However such building groups are not regular building units in peptide synthesis and as such are very expensive compared to Fmoc-Gly-OH or Fmoc-Gly-Gly-OH which makes their use limited to small scale preparations but inefficient on large production scale.

Thus the production of a high purity peptide product is a highly desired but difficult to achieve goal.

### SUMMARY OF THE INVENTION

In one embodiment, the present invention provides a method for the purification of a peptide containing a poly-Gly sequence from Gly addition or Gly deletion byproducts comprising the steps of:

(a) preparing a protected peptide attached to a resin using Fmoc-Gly-Gly-OH units for assembly of the poly-Gly segment;

(b) cleaving the protected peptide obtained in step (a) from the resin with an acidic composition to produce an unprotected or semi-protected crude peptide;

(c) isolating the unprotected or semi-protected crude peptide from the acidic composition; and

(d) removing any remaining protecting groups from the isolated crude peptide to obtain an unprotected crude peptide.

### DETAILED DESCRIPTION OF THE INVENTION

For purposes of clarity and as an aid in understanding the invention, as disclosed and claimed herein, the following terms and abbreviations are defined below:
Boc—t-Butyloxycarbonyl
DCM—dichloromethane
DDM dodecylmercaptane
DMF—dimethylformamide
EDT—ethanedithiol
Fmoc—9-fluorenylmethoxycarbonyl
HBTU—2-(1H-Benzotriazole-1-yl)-1,1,3,3-tetramethyluronium hexafluorophosphate
HOBt—N-hydroxybenzotriazole
MTBE—Methyltertiarybutylether
Pbf—pentamethyldihydrobenzofuransulfonyl
TFA—trifluoroacetic acid
TIS—triisopropylsilane
Trt—trityl

As used herein, “having a purity by HPLC” refers to purity from “related peptides” analysis. “Related peptides” are impurities such as truncated peptides, which may be present due to incomplete synthesis of the peptides, and other potential peptide derivatives obtained from peptide synthesis.

As used herein, “a Gly deletion byproduct” refers to a by-product which is performed due to an incomplete addition of a glycine amino acid.

As used herein, “a Gly addition byproduct” refers to a by-product which is performed due to addition of additional Gly residue over the required sequence.

Sequential preparation of Bivalirudin using Fmoc chemistry results in formation of two main impurities related to the synthesis of Gly-Gly-Gly-Gly segment: [des-Gly]-Bivalirudin and [+Gly]-Bivalirudin. Formation of these impurities can be efficiently reduced by use of two cycles of Fmoc-Gly-Gly-OH coupling instead of four couplings of Fmoc-Gly-OH. It is believed that because Fmoc-Gly-Gly-OH, not Fmoc-Gly-OH units, are added to the growing peptide, addition or deletion of these units will no longer result in a peptide containing a 3 or 5 Glycine sequence ([des-Gly]-Bivalirudin and [+Gly]-Bivalirudin, respectively), but rather in a 2 or 6 Glycine sequence which are more easily separated from the peptide. The amount of the impurities are also reduced because when Fmoc-Gly-Gly-OH units are used, less cycles are required in order to synthesize the 4 Gly sequence. Therefore deletion/addition of units occur less frequently.

When using RP-HPLC, both impurities elute very close to the Bivalirudin peptide, thus imposing a severe problem in the purification of the peptide using preparative RP-HPLC. As a result, a relatively high content of the above impurities are present in the final product when using the sequential synthesis method.

In one embodiment, the present invention provides a method for the purification of a peptide containing a poly-Gly sequence from Gly addition or Gly deletion byproducts comprising the steps of:

(a) preparing a protected peptide attached to a resin using Fmoc-Gly-Gly-OH units for assembly of the poly-Gly segment;

(b) cleaving the protected peptide obtained in step (a) from the resin with an acidic composition to produce an unprotected or semi-protected crude peptide;

(c) isolating the unprotected or semi-protected crude peptide from the acidic composition; and

(d) removing any remaining protecting groups from the isolated crude peptide to obtain an unprotected crude peptide.

The method can further comprise the steps of:

(a) purifying the unprotected crude peptide by chromatography to obtain a peptide solution comprising a high purity peptide, and

(b) drying the peptide solution to obtain a peptide containing a poly-Gly sequence.

Preferably, the obtained peptide containing a poly-Gly sequence has a purity of at least about 98.5% by HPLC and/or less than about 0.5% of a Gly deletion by-product and/or less than about 0.5% of a Gly addition by-product.

More preferably, the obtained peptide containing a poly-Gly sequence has a purity of at least about 99% by HPLC and/or less than about 0.2% of a Gly deletion by-product and/or less than about 0.2% of a Gly addition by-product.

In a preferred embodiment, the present invention provides a process for the preparation of Bivalirudin comprising:

(a) preparing a protected Bivalirudin peptide attached to a resin using Fmoc-Gly-Gly-OH units for assembly of the Gly-Gly-Gly-Gly segment;

(b) cleaving the protected Bivalirudin obtained in step (a) from the resin with an acidic composition to produce an unprotected or semi-protected crude Bivalirudin;

(c) isolating the unprotected or semi-protected crude Bivalirudin from the acidic composition; and

(d) removing any remaining protecting groups from the isolated crude Bivalirudin to obtain an unprotected crude Bivalirudin peptide.

The method can further comprise the steps of:

(a) purifying the crude Bivalirudin peptide by chromatography to obtain a peptide solution comprising a high purity Bivalirudin peptide, and

(b) drying the peptide solution to obtain Bivalirudin of high purity, preferably through lyophilization.

Preferably, the obtained Bivalirudin containing a Gly-Gly-Gly-Gly sequence has a purity of at least about 98.5% by HPLC and/or less than about 0.5% of a [des-Gly]-Bivalirudin and/or less than about 0.5% of [+Gly]-Bivalirudin.

More preferably, the obtained Bivalirudin containing a Gly-Gly-Gly-Gly sequence has a purity of at least about 99% by HPLC and/or less than about 0.2% of a [des-Gly]-Bivalirudin and/or less than about 0.2% of [+Gly]-Bivalirudin.

A suitable method for the determination of the purity of the Bivalirudin peptide includes, but is not limited to, using HPLC. A preferred method of determining the purity of the Bivalirudin peptide comprises a HPLC system with a RP and HILIC columns and MS analysis.

Optionally, the obtained Bivalirudin may be subjected to a counterion exchange to obtain a Bivalirudin-TFA salt by any conventional method.

The peptides synthesized by the process of the invention are preferably prepared using Fmoc-chemistry. Suitable methods include, but not limited to, using solid-phase synthesis with an acid-labile resin wherein the first amino acid is attached to the resin via an acid labile ester linkage and using Fmoc-Gly-Gly-OH unit for preparation of the Gly-Gly-Gly-Gly segment during the peptide assembly.

Suitable acid-labile resins for use in the process include, but are not limited to, acid-labile resins such as Wang resin, chlorotrityl resins such as 2-Cl-Trt-Cl resin, HMPB-BHA resin, Rink acid resin, Rink amide resin, Rink amide AM resin, Rink amide MBHA resin, or NovaSyn TGT alcohol resin. In a preferred embodiment the acid-labile resin is Wang resin or 2-Cl-Trt-Cl resin.

Coupling agents include, but are not limited to, 2-(1H-benzotriazole-1-yl)-1,1,3,3-tetramethyluronium tetrafluoroborate (TBTU), DCC, DIC, HBTU, BOP, or PyBOP. Optionally, when the coupling reagent is TBTU, it may be used together with HOBT to suppress racemization.

Coupling of a protected peptide is carried out in a solvent such as DMF. This coupling solvent may also contain an organic base such as for example diisopropylethylamine (DIPEA) or Collidine. The carboxylic group of the Fmoc-Gly-Gly-OH or Fmoc amino acids can be activated by a suitable method prior to introduction to the amino compound or in-situ in the reaction mixture. A step of washing may be performed after steps which include a chemical reaction, such as a coupling reaction, for removal of unreacted materials and other by-products. Suitable solvents for use in the washing steps of this process include, but are not limited to, dimethylformamide (DMF), dichloromethane (DCM), methanol (MeOH), ethanol, acetonitrile, methyltertbutyl ether (MTBE), or isopropanol (IPA). Water may also be used.

Suitable protecting groups for the terminal α-amine acid residue include, but are not limited to, 9-fluorenylmethoxycarbonyl (Fmoc) and BOC. The more stable protecting group used on the other functional residues of the amino acids includes, but is not limited to Pbf, tBu, Trt, and Boc. Preferably Pbf for Arg residues and tBu, Trt and Boc for all other amino acid residues.

In a preferred embodiment the hyper acid-labile resin used in the process of the invention is Wang or 2-Cl-Trt-Cl resin.

Cleavage of the peptide from the resin as described in step b) and removal of the protecting groups as described in step d) may be performed using any conventional method. For example, one method includes, but is not limited to, a TFA based cocktail that contains in addition to TFA several scavengers such as EDT, DDM, phenol, thioanisole, and water.

The terminal amino acid residue Fmoc protecting group is removed by any known method, such as reaction with a piperidine solution in DMF. However, one of ordinary skill in the art may substitute the reagents in the deprotection solution with other suitable reagents, such as DBU, DBU/piperidine, and diethylamine. Cleavage of the acid-labile protecting groups from the peptide may be affected by addition of a strong acidic composition. The acidic composition is preferably based on an acidic material such as TFA, and contains scavenger reagents including, but not limited to, ethanedithiol (EDT), thioanisole, TIS, DDM, phenol, m-cresol, and water. The relative ratio of acidic material to scavenger to water may be from about 85% to about 99% acidic material, from about 0.1% to about 15% scavenger, and from about 0.1% to about 15% water by weight. A preferred acidic composition comprises about 95% TFA, about 2.5% EDT, and about 2.5% water.

Isolation of the unprotected or semi-protected peptide as described in step (c) may be performed by any conventional method such as by precipitation, crystallization, extraction or chromatography. Preferably, isolation of the unprotected or semi-protected peptide is performed by precipitation.

Optionally, when preparing Bivalirudin, the isolation of the unprotected or semi-protected crude Bivalirudin in step (c) may be performed by precipitation of the crude peptide from a mixture of the crude peptides and a solvent comprises or consists of a lower alkyl ether. Preferably, the lower alkyl ether is diethylether or MTBE.

The crude peptide product may be purified by any known method. Preferably, the peptide is purified using HPLC on a reverse phase (RP-HPLC) column using a binary gradient consisting of water and one or more organic solvents, including but not limited to acetonitrile, methanol, n-propanol, or IPA. The resulting purified product is dried and may be lyophilized or spray-dried.

Advantages of preferred processes of the invention are that all synthetic steps are performed under mild conditions, a low amount of byproducts are produced, and/or a high yield and/or a high purity of the final Bivalirudin peptide product. Other advantages is that preferred embodiments use commercially available, inexpensive starting materials.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

### Example 1

### Preparation of Bivalirudin by Sequential Solid Phase Synthesis on CTC Resin

Synthesis of the peptide sequence was carried out by a stepwise Fmoc SPPS (solid phase peptide synthesis) procedure starting with H-Leu-2-Cl-Trt resin (4.5 Kg). The resin was washed by several washings with DMF and after the washing the second amino acid (Fmoc-Tyr(tBu)) was introduced to start the first coupling step. The Fmoc protected amino acid was activated in situ using TBTU/HOBt (N-hydroxybenzotriazole) and subsequently coupled to the resin for 50 minutes. Diisopropylethylamine was used during coupling as an organic base. Completion of the coupling was indicated by a Ninhydrine test. After washing of the resin, the Fmoc protecting group on the α-amine was removed with 20% piperidine in DMF for 20 min. These steps were repeated each time with another amino acid according to peptide sequence.

All amino acids used were Fmoc-N^{α} protected except the last amino acid in the sequence, Boc-D-Phe. Trifunctional amino acids were side chain protected as follows: Ser(tBu), Arg(Pbf), Tyr(tBu), Asp(OtBu) and Glu(OtBu). Fmoc-Gly-Gly-OH was preactivated by DIC/HOBt and used for synthesis of Gly-Gly-Gly-Gly segment. Three equivalents of the activated amino acids were used in the coupling reactions. At the end of the synthesis the peptide-resin was washed with DMF, followed by MeOH, and dried under vacuum to obtain 16.6 Kg dry peptide-resin.

The cleavage of the peptide from the resin with simultaneous deprotection of the protecting groups was performed as following: a) 16.6 Kg peptide resin obtained as described above were added to the reactor containing a cold solution of 95% TFA, 2.5% TIS, 2.5% EDT; b) the mixture was mixed for 2 hours at room temperature; c) the product was precipitated by the addition of 10 volumes of ether (MTBE), filtered and dried in vacuum to obtain 7.6 Kg crude product.

The crude peptide (7.6 Kg) obtained above, was dissolved in aqueous solution of acetonitrile. The resulting solution was loaded on a C₁₈ RP-HPLC column and purified to obtain fractions containing Bivalirudin at a purity of >97.5%. The pure fractions were collected and lyophilized to obtain a final dry peptide (1.7 Kg) which was >99.0% pure (HPLC). It contained <0.5% [Asp⁹-Bivalirudin], <0.2% [des-Gly]-Bivalirudin, <0.2% [+Gly]-Bivalirudin and not more than 0.5% of any other impurity.

### Example 2

### Preparation of Bivalirudin by Sequential Solid Phase Synthesis on Wang Resin

Synthesis of the peptide sequence was carried out by a stepwise Fmoc SPPS (solid phase peptide synthesis) procedure starting with H-Leu-Wang resin (250 g). The resin was washed several times with DMF, and after the washing the second amino acid (Fmoc-Tyr(tBu)) was introduced to start the first coupling step. The Fmoc protected amino acid was activated in situ using TBTU/HOBt (N-hydroxybenzotriazole) and subsequently coupled to the resin for 50 minutes. Diisopropylethylamine was used during coupling as an organic base. Completion of the coupling was indicated by a Ninhydrine test. After washing of the resin, the Fmoc protecting group on the α-amine was removed with 20% piperidine in DMF for 20 min. These steps were repeated each time with another amino acid according to peptide sequence.

All amino acids used were Fmoc-N^{α} protected except the last amino acid in the sequence, Boc-D-Phe. Trifunctional amino acids were side chain protected as follows: Ser(tBu), Arg(Pbf), Tyr(tBu), Asp(OtBu) and Glu(OtBu). Fmoc-Gly-Gly-OH was preactivated by DIC/HOBt and used for synthesis of Gly-Gly-Gly-Gly segment. Three equivalents of the activated amino acids were used in the coupling reactions. At the end of the synthesis the peptide-resin was washed with DMF, followed by MeOH, and dried under vacuum to obtain 904 g dry peptide-resin.

The cleavage of the peptide from the resin with simultaneous deprotection of the protecting groups was performed as following: a) 904 g peptide resin obtained as described above were added to the reactor containing a cold solution of 95% TFA, 2.5% TIS, 2.5% EDT; b) the mixture was mixed for 2 hours at room temperature; c) the product was precipitated by the addition of 10 volumes of ether (MTBE), filtered and dried in vacuum to obtain 470 g crude product.

The crude peptide (470 g) obtained above, was dissolved in aqueous solution of IPA. The resulting solution was loaded on a C₁₈ RP-HPLC column and purified to obtain fractions containing Bivalirudin at a purity of >97.5%. The pure fractions were collected and lyophilized to obtain a final dry peptide (92 g) which was >99.0% pure (HPLC). It contained <0.5% [Asp⁹-Bivalirudin], <0.2% [des-Gly]-Bivalirudin, <0.2% [+Gly]-Bivalirudin and not more than 0.5% of any other impurity.

### Example 3

### Preparation of Bivalirudin by Sequential Solid Phase Synthesis on CTC Resin—Without Use of Fmoc-Gly-Gly-OH

Synthesis of the peptide sequence was carried out by a stepwise Fmoc SPPS (solid phase peptide synthesis) procedure starting with H-Leu-2-Cl-Trtl resin (3.0 Kg). The resin was washed several times with DMF, and after the washing the second amino acid (Fmoc-Tyr(tBu)) was introduced to start the first coupling step. The Fmoc protected amino acid was activated in situ using TBTU/HOBt (N-hydroxybenzotriazole) and subsequently coupled to the resin for 50 minutes. Diisopropylethylamine was used during coupling as an organic base. Completion of the coupling was indicated by a Ninhydrine test. After washing of the resin, the Fmoc protecting group on the α-amine was removed with 20% piperidine in DMF for 20 min. These steps were repeated each time with another amino acid according to peptide sequence.

All amino acids used were Fmoc-N^{α} protected except the last amino acid in the sequence, Boc-D-Phe. Trifunctional amino acids were side chain protected as follows: Ser(tBu), Arg(Pbf), Tyr(tBu), Asp(OtBu) and Glu(OtBu). Three equivalents of the activated amino acids were used in the coupling reactions. At the end of the synthesis the peptide-resin was washed with DMF, followed by MeOH, and dried under vacuum to obtain 10.5 Kg dry peptide-resin.

The cleavage of the peptide from the resin with simultaneous deprotection of the protecting groups was performed as following: a) 10.5 Kg peptide resin obtained as described above were added to the reactor containing a cold solution of 95% TFA, 2.5% TIS, 2.5% EDT; b) the mixture was mixed for 2 hours at room temperature; c) the product was precipitated by the addition of 10 volumes of ether (MTBE), filtered and dried in vacuum to obtain 4.9 Kg crude product.

The crude peptide (4.9 Kg) obtained above, was dissolved in aqueous solution of acetonitrile. The resulting solution was loaded on a C₁₈ RP-HPLC column and purified to obtain fractions containing Bivalirudin at a purity of >97.5%. The pure fractions were collected and lyophilized to obtain a final dry peptide (960 g) which was >97.5% pure (HPLC). It contained <0.5% [Asp⁹-Bivalirudin], 0.4% [des-Gly]-Bivalirudin, 0.5% [+Gly]-Bivalirudin and not more than 0.5% of any other impurity.

1. A method for preparing a peptide containing a poly-Gly sequence and having low amounts of Gly addition or Gly deletion byproducts comprising:
(a) preparing a protected poly-Gly-containing peptide attached to a resin using Fmoc-Gly-Gly-OH units for assembly of the poly-Gly segment; and
(b) cleaving the protected peptide obtained in step (a) from the resin with an acidic composition to produce an unprotected or semi-protected crude peptide.

2. The method of claim 1, further comprising:
isolating the unprotected or semi-protected crude peptide from the acidic composition.

3. The method of claim 1, further comprising:
when the product of step (b) includes semi-protected crude peptide, removing protecting groups from the semi-protected crude peptide to obtain an unprotected crude peptide.

4. The method of claim 1, further comprising:
isolating the peptide from Gly addition byproducts, Gly deletion byproducts, or both, thereby forming a purified peptide.

5. The method of claim 1, wherein the purified peptide has a purity of at least about 98.5%.

6. The method of claim 4, wherein the purified peptide contains less than about 0.5% of Gly deletion byproducts.

7. The method of claim 4, wherein the purified peptide contains less than about 0.5% of Gly addition byproducts.

8. The method of claim 1, wherein the peptide containing a poly-Gly sequence is Bivalirudin.

9. A purified peptide containing a poly-Gly sequence having a purity of at least about 98.5%.

10. The purified peptide of claim 9 having less than about 0.5% of Gly deletion byproducts.

11. The purified peptide of claim 9 having less than about 0.5% of Gly addition byproducts.

12. The purified peptide of claim 9, wherein the peptide containing a poly-Gly sequence is Bivalirudin.

13. A method for preparing Bivalirudin comprising:
(a) preparing a protected Bivalirudin peptide attached to a resin using Fmoc-Gly-Gly-OH units for assembly of the Gly-Gly-Gly-Gly segment;
(b) cleaving the protected Bivalirudin obtained in step (a) from the resin with an acidic composition to produce an unprotected or semi-protected crude Bivalirudin;
(c) isolating the unprotected or semi-protected crude Bivalirudin from the acidic composition; and optionally
(d) removing remaining protecting groups from the isolated crude Bivalirudin to obtain an unprotected crude Bivalirudin peptide.

14. The method of claim 13, further comprising:
purifying the crude Bivalirudin peptide by chromatography to obtain a purified Bivalirudin peptide solution; and
drying the purified Bivalirudin peptide solution to obtain purified Bivalirudin.

15. The method of claim 14, wherein the drying step comprises lyophilizing the solution.

16. The method of claim 14, wherein the purified Bivalirudin has a purity of at least about 98.5%.

17. The method of claim 14, wherein the purified Bivalirudin contains less than about 0.5% of [+Gly]-Bivalirudin.

18. The method of claim 14, wherein the purified Bivalirudin contains less than about 0.5% of [des-Gly]-Bivalirudin.

19. (canceled)


SEQUENCE LISTING (8 sequences)

SEQ ID NO: 1 - protein, 28 aa, Artificial Sequence
  Synthetic peptide
SLRRSSCFGGRMDRIGAQSGLGCNSFRY

SEQ ID NO: 2 - protein, 35 aa, Artificial Sequence
  Synthetic Peptide
RREAEDLQVGQVELGGGPGAGSLQPLALEGSLQKR

SEQ ID NO: 3 - protein, 13 aa, Artificial Sequence
  Synthetic Peptide
YGGFLRRQFKVVT

SEQ ID NO: 4 - protein, 16 aa, Artificial Sequence
  Synthetic Peptide
YGGFMTSEKSQTPLVT

SEQ ID NO: 5 - protein, 24 aa, Artificial Sequence
  Synthetic Peptide
VLGGGVALLRVIPALDSLTPANED

SEQ ID NO: 6 - protein, 12 aa, Artificial Sequence
  Synthetic Peptide
GGGCYFQNCPKG

SEQ ID NO: 7 - protein, 39 aa, Artificial Sequence
  Synthetic Peptide
HGEGTFTSDLSKQMEEEAVRLFIEWLKNGGPSSGAPPPS

SEQ ID NO: 8 - protein, 20 aa, Artificial Sequence
  Synthetic Peptide
FPRPGGGGNGDFEEIPEEYL